(19) United States
(12) Patent Application Publication
(10) Pub. No.: US 2011/0117210 A1
(43) Pub. Date: May 19, 2011

(54) Therapeutic treatment of human cancers using simple salts of zinc

(76) Inventor: Andrey Ugolkov, Buffalo Grove, IL (US)

(21) Appl. No.: 12/590,951

(22) Filed: Nov. 17, 2009

(51) Int. Cl. A61K 33/30; A61P 35/00; A61K 31/315

(52) U.S. Cl. 424/643; 514/494

(57) ABSTRACT

The present invention is a non-topical method of therapeutically treating human cancer patients which comprises an administration of an anti-cancer medicament comprising at least one simple organic or inorganic salt of zinc in at least a minimally effective concentration to suppress malignant tumor growth and induce tumor regression in-vivo. Administration can be performed by oral, parenteral, and/or body cavity routings; and the therapeutic treatment method is effective for the treatment of a diverse range of primary human cancers and metastatic diseases.

### FIELD OF THE INVENTION

The present invention is concerned with therapeutic treatment methods effective against recognized both solid and disseminated forms of human cancers including hematological malignancies; and is particularly directed to the use of simple organic and inorganic salts of zinc (“Zn”) administered in at least minimally effective concentrations as a therapeutic treatment regimen which is efficacious in-vivo against recognized forms of human cancer and metastatic disease.

### BACKGROUND OF THE INVENTION

Zinc is an essential mineral that is naturally present in some foods, is purposefully added to others, and is also available for ingestion as a dietary supplement. Zinc is also found in many cold lozenges and some over-the-counter drugs sold as cold remedies.

A wide variety of consumable foods contain zinc. Oysters contain more zinc per serving than any other food, but red meat and poultry continue to provide the majority of zinc in the American diet. Other good food sources include beans, nuts, certain types of seafood (such as crab and lobster), whole grains, fortified breakfast cereals, and dairy products.

Note however that food Phytates (phosphorus compounds such as inositol hexaphosphate)—which are present in whole-grain breads, cereals, legumes, and other foods—directly bind zinc ions and inhibit its absorption in-vivo. Thus, the bioavailability of zinc from grains and plant foods is often less than that obtained from animal foods, although many grain- and plant-based foods are still considered to be good dietary sources of zinc.

Also, many persons today routinely take zinc containing compounds as a dietary supplement. Such dietary supplements often contain several forms of zinc, including zinc gluconate, zinc sulfate, and zinc acetate. However, the percentage of elemental zinc present in these varies by form. For example, approximately 23% of zinc sulfate in the supplement is elemental zinc; thus, 220 mg of each zinc sulfate supplement tablet or capsule contains only 50 mg of elemental zinc. The true elemental zinc content typically appears in the Supplement Facts panel on the supplement container. Research has not determined whether differences in absorption, bioavailability, or tolerability exist among the various forms of zinc supplements. In addition to standard tablets and capsules, some zinc-containing cold lozenges are labeled as dietary supplements.

### Other Common Sources of Zinc

Zinc is often present in several products sold over the counter as natural medicines for colds; and typically exists in the form of lozenges, nasal sprays, and ingestible gels. Numerous case reports of anosmia (loss of smell), in some cases long-lasting or permanent, have occurred from the use of zinc-containing nasal gels or sprays [see for example, Jafek B W, Linschoten M R, Murrow B W. Anosmia after intranasal zinc gluconate use. Am J Rhinol 2004;18:137-141 & Alexander T H, Davidson T M. Intranasal zinc and anosmia: the zinc-induced anosmia syndrome. Laryngoscope 2006;116:217-20].

These reports have raised serious questions about the safety of intranasal zinc. In fact, during June 2009, the FDA warned consumers to stop using three zinc-containing intranasal products because they might cause anosmia [U.S. Food and Drug Administration. Warnings on Three Zicam Intranasal Zinc Products. [http://www.fda.gov/ForConsumers/Consumers_Updates/ucm166931.htm]. As a consequence of this FDA warning, the manufacturer of these zinc intranasal sprays has voluntarily withdrawn these products from the marketplace.

### A. The Recognized Role of Zinc Ions in Human Metabolism

Zinc ions are essential to human metabolism and serve multiple biological functions in humans that are conventionally classified as catalytic, structural, or regulatory. Zinc ions as such are needed for the proper growth and maintenance of the human body. In particular, zinc is needed for proper immune function, wound healing, blood clotting, thyroid function, and much more. Moreover, zinc is necessary for the functioning of more than 300 different enzymes and plays a vital role in an enormous number of different biological processes.

For example, zinc is a necessary cofactor for the antioxidant enzyme superoxide dismutase (SOD), and is also required to be present in a number of enzymatic reactions involved in carbohydrate and protein metabolism. Long recognized zinc immune-enhancing activities today include the regulation of T lymphocytes, CD4, natural killer cells, and interleukin II; and, zinc ions are said to possess some antiviral activity.

In addition, zinc has been shown to play a role in wound healing, especially following burns or surgical incisions. Zinc is also necessary for the maturation of sperm and normal fetal development; and is involved in sensory perception (taste, smell, and vision) and controls the release of stored vitamin A from the liver. Within the endocrine system, zinc has been shown to regulate insulin activity and promote the conversion thyroid hormones thyroxine to triiodothyronine.

As evidence of these recognized human metabolic functions, the following publications are presented as representative and exemplary studies of zinc ion activity: Prasad AS. Zinc: an overview. Nutrition 1995;11:93-9. Heyneman C A. Zinc deficiency and taste disorders. Ann Pharmacother 1996;30:186-7. Simmer K, Thompson R P. Zinc in the fetus and newborn. Acta Paediatr Scand Suppl 1985;319:158-63. Fabris N, Mocchegiani E. Zinc, human diseases and aging. Aging (Milano) 1995;7:77-93. Maret W, Sandstead H H. Zinc requirements and the risks and benefits of zinc supplementation. J Trace Elem Med Biol 2006;20:3-18. Prasad A S, Beck F W, Grabowski S M, Kaplan J, Mathog R H. Zinc deficiency: changes in cytokine production and T-cell subpopulations in patients with head and neck cancer and in noncancer subjects. Proc Assoc Am Physicians 1997;109:68-77. Rink L, Gabriel P. Zinc and the immune system. Proc Nutr Soc 2000;59:541-52.

### Some Speculative Uses for Zinc Ions

As reported and based on some publically available scientific evidence, zinc ions may also be effective in the treatment of (childhood) malnutrition, acne vulgaris, peptic ulcers, leg ulcers, infertility, Wilson's disease, herpes, and taste or smell disorders. Zinc has also gained some popularity for its alleged role in the prevention of the common cold.

The use of zinc within these particular medical circumstances was previously and remains today very controversial—because the results of different published studies present either directly contradictory information, and/or the methodological quality of the published studies does not allow a medical practitioner to draw a confident conclusion regarding the actual value (if any) of zinc in these pathological states.

For those persons wishing more information and details about such speculative applications, a representative listing of relevant publications includes: Hulisz D. Efficacy of zinc against common cold viruses: an overview. J Am Pharm Assoc (2003) 2004;44:594-603. Caruso T J, Prober C G, Gwaltney J M Jr. Treatment of naturally acquired common colds with zinc: a structured review. Clin Infect Dis 2007;45:569-74. Prasad A S, Beck F W, Bao B, Snell D, Fitzgerald J T. Duration and severity of symptoms and levels of plasma interleukin-1 receptor antagonist, soluble tumor necrosis factor receptor, and adhesion molecules in patients with common cold treated with zinc acetate. Infect Dis 2008 ;197:795-802. Turner R B, Cetnarowski W E. Effect of treatment with zinc gluconate or zinc acetate on experimental and natural colds. Clin Infect Dis 2000;31:1202-8. Eby G A, Halcomb W W. Ineffectiveness of zinc gluconate nasal spray and zinc orotate lozenges in common-cold treatment: a double-blind, placebo-controlled clinical trial. Altern Ther Health Med 2006;12:34-8. Marshall I. Zinc for the common cold. Cochrane Database Syst Rev 2000;(2):CD001364.

### B. Recommended Zinc Intakes for Healthy Individuals

Daily human intake recommendations for zinc are provided in the Dietary Reference Intakes (“DRIs”) developed by the Food and Nutrition Board (“FNB”) at the Institute of Medicine of the National Academies [see Institute of Medicine, Food and Nutrition Board. Dietary Reference Intakes for Vitamin A, Vitamin K, Arsenic, Boron, Chromium, Copper, Iodine, Iron, Manganese, Molybdenum, Nickel, Silicon, Vanadium, and Zinc. Washington, D.C.: National Academy Press, 2001]. The DRI is a set of reference values used for planning and assessing nutrient intakes of healthy (medically normal) people. These intake values, which vary by age and gender, include the following:
Recommended Dietary Allowance (RDA): average daily level of intake sufficient to meet the nutrient requirements of nearly all (97%-98%) healthy individuals.
Adequate Intake (AI): established when evidence is insufficient to develop an RDA and is set at a level assumed to ensure nutritional adequacy.
Tolerable Upper Intake Level (UL): maximum daily intake unlikely to cause adverse health effects.

The current RDAs for zinc are listed by Table A below. For infants aged 0 to 6 months, the FNB established an Adequate Intake for zinc that is equivalent to the mean intake of zinc in healthy, breastfed infants.

TABLE A
Recommended Dietary Allowances (RDAs) for Zinc^{#}

| Age | Male | Female | Pregnancy | Lactation |
|---|---|---|---|---|
| Birth to 6 months | 2 mg* | 2 mg* | | |
| 7 months to 3 years | 3 mg | 3 mg | | |
| 4 to 8 years | 5 mg | 5 mg | | |
| 9 to 13 years | 8 mg | 8 mg | | |
| 14 to 18 years | 11 mg | 9 mg | 13 mg | 14 mg |
| 19+ years | 11 mg | 8 mg | 11 mg | 12 mg |

*Adequate Intake (AI)
^{#}Reproduced from: Institute of Medicine, Food and Nutrition Board. Dietary Reference Intakes for Vitamin A, Vitamin K, Arsenic, Boron, Chromium, Copper, Iodine, Iron, Manganese, Molybdenum, Nickel, Silicon, Vanadium, and Zinc. Washington, DC: National Academy Press, 2001

### C. The Risks and Dangers of Ingesting Zinc

Zinc is ubiquitous in nature and exists in many forms. The ingestion of some forms of zinc causes the creation of toxic zinc salts in-situ within the acidic gastric environment of the body; while ingestion of one or more zinc salts introduces elemental zinc into the gastro-intestinal tract for direct absorption.

Zinc toxicity has been documented in living humans as well as in a wide range of large and small animals. Zinc toxicity is most commonly seen in pet dogs, and is believed due at least in part to a higher degree of dietary indiscretion and greater levels of exposure to ordinary zinc-containing articles. Common examples of zinc-containing articles include batteries, automotive parts, paints, zinc-oxide creams, herbal supplements, zippers, board-game pieces, screws and nuts on pet carriers, and the coatings on galvanized metals used as pipes and cookware.

### Pathogenesis

If zinc metal is ingested, the very low pH in the stomach will cause the formation of soluble zinc salts in-situ. These zinc salts are then absorbed from the duodenum; and are rapidly distributed to the liver, kidneys, prostate, muscles, bones, and pancreas of the living subject.

If one or more zinc salts are ingested, these salts may cause direct irritant and corrosive effects on living organs and tissues; may interfere with the metabolism of other ions such as copper, calcium, and iron; and may inhibit erythrocyte production and function.

Curiously, the mechanisms by which zinc ions exert their toxic effects in-vivo are still not completely understood today. Nevertheless, what is factually documented and medically known is that the median lethal dose (LD₅₀) of ingested zinc salts in cases of acute toxicity is ˜100 mg/kg. Furthermore, diets containing high levels of zinc (i.e., >2,000 ppm) are known to cause chronic zinc toxicosis.

### Clinical Signs and Lesions

Clinical signs of zinc toxicity will vary based on the duration and degree of zinc ion exposure. The classical signs of zinc toxicity will progress from anorexia and vomiting, to more advanced symptoms such as diarrhea, lethargy, icterus, shock, intravascular hemolysis, hemoglobinuria, cardiac arrhythmias, and even seizures. Large animals will often show decreases in weight gain and milk production; and lameness has also been reported in foals secondary to epiphyseal swelling.

Major histopathologic findings of zinc toxicity include hepatocellular centrolobular necrosis with hemosiderosis and vacuolar degeneration, renal tubular necrosis with hemoglobin casts, and pancreatic duct necrosis with fibrosis of the interlobular fat.

### Human Zinc Toxicity

The risk and dangers of zinc toxicity in humans can occur in both acute and chronic forms. Acute adverse effects of high zinc intake can include nausea, vomiting, loss of appetite, abdominal cramps, diarrhea, and headaches. In one particular instance of human acute toxicity, severe nausea and vomiting occurred within 30 minutes of ingesting 4 grams of zinc gluconate, equaling 570 milligrams of elemental zinc [Lewis M R & Kokan L., Zinc gluconate: acute ingestion, J Toxicol Clin Toxicol 1998;36:99-101].

It is now well documented also that human intakes of 150-450 mg of zinc per day have been associated with such chronic effects as low copper status, altered iron function, reduced immune function, and reduced levels of high-density lipoproteins [Hooper P L, Visconti L, Garry P J, Johnson G E. Zinc lowers high-density lipoprotein-cholesterol levels. J Am Med Assoc 1980;244:1960-1961]. Similarly, reductions in a copper-containing enzyme (a marker of copper status) have been reported with even moderate zinc intakes of approximately 60 mg/day for up to 10 weeks [Institute of Medicine, Food and Nutrition Board. Dietary Reference Intakes for Vitamin A, Vitamin K, Arsenic, Boron, Chromium, Copper, Iodine, Iron, Manganese, Molybdenum, Nickel, Silicon, Vanadium, and Zinc. Washington, D.C.: National Academy Press, 2001].

Physicians are also aware that the doses of zinc used in the AREDS study—80 mg per day of zinc in the form of zinc oxide for 6.3 years, on average—have been directly correlated with a significant increase in hospitalizations for genitourinary causes. This confirms and reinforces the generally accepted view that chronically high intakes of zinc will adversely affect at least some aspects of urinary physiology[Johnson A R, Munoz A, Gottlieb J L, Jarrard D F. High dose zinc increases hospital admissions due to genitourinary complications. J Urol 2007;177:639-43].

In short, the published scientific and medical literature has documented and established the following facts:
Human zinc toxicity can occur in both acute and chronic forms. Toxicity from chronic ingestion of zinc usually occurs as copper deficiency [Toxbase, National Poisons Information Service].
Humans ingesting 200 to 800 mg/day of zinc may cause abdominal pain, nausea, vomiting and diarrhea. Other reported toxic effects include lethargy, anemia and dizziness.
Human intake of zinc ranging from 100 to 150 mg/day may interfere with copper metabolism and cause low copper status, reduced iron function, red blood cell microcytosis, neutropenia, reduced immune function and reduced levels of high-density lipoproteins [Food Standards Agency, Zinc].

### Human Tolerable Dosages of Zinc

All the reported instances of human zinc toxicity clearly illustrate the risks and dangers of ingesting too much zinc. In order to avoid such risks, the Food and Nutrition Board (at the Institute of Medicine of the National Academies) has established maximum tolerable Upper Intake Levels (or “ULs”) for medically normal humans ingesting zinc. These maximal tolerable doses of zinc for medically normal human are presented by Table B below. Accordingly, it is generally recognized by medical practitioners that long-term human intakes above the tolerable Upper Intake Levels (as given by Table B) will markedly increase the risk of adverse health effects in humans.

TABLE B
Tolerable Upper Intake Levels for Zinc⁺

| Age | Male | Female | Pregnant | Lactating |
|---|---|---|---|---|
| 0 to 6 months | 4 mg | 4 mg | | |
| 7 to 12 months | 5 mg | 5 mg | | |
| 1 to 3 years | 7 mg | 7 mg | | |
| 4 to 8 years | 12 mg | 12 mg | | |
| 9 to 13 years | 23 mg | 23 mg | | |
| 14 to 18 years | 34 mg | 34 mg | 34 mg | 34 mg |
| 19+ years | 40 mg | 40 mg | 40 mg | 40 mg |

⁺Reproduced from: Institute of Medicine, Food and Nutrition Board. Dietary Reference Intakes for Vitamin A, Vitamin K, Arsenic, Boron, Chromium, Copper, Iodine, Iron, Manganese, Molybdenum, Nickel, Silicon, Vanadium, and Zinc. Washington, DC: National Academy Press, 2001.

### D. Zinc Interactions with Commonly Prescribed Medications

It has been long recognized that zinc ions have the potential to interact with several types of commonly prescribed medications. A few examples of such interactions are described below.

### Antibiotics

Both quinolone antibiotics (such as Cipro®) and tetracycline antibiotics (such as Achromycin® and Sumycin®) interact with zinc in the gastrointestinal tract, thereby inhibiting the absorption of both zinc and the antibiotic [Lomaestro B M, Bailie G R. Absorption interactions with fluoroquinolones. 1995 update. Drug Saf 1995;12:314-33. Penttilä O, Hurme H, Neuvonen P J. Effect of zinc sulphate on the absorption of tetracycline and doxycycline in man. Eur J Clin Pharmacol 1975;9:131-4]. Taking the antibiotic at least 2 hours before or 4-6 hours after taking a zinc supplement minimizes this interaction [Natural Medicines Comprehensive Database. Zinc. [http://www.naturaldatabase.com/].

### Penicillamine

Zinc can reduce the absorption and action of penicillamine, a drug used to treat rheumatoid arthritis [Brewer G J, Yuzbasiyan-Gurkan V, Johnson V, Dick R D, Wang Y. Treatment of Wilson's disease with zinc: XI. Interaction with other anticopper agents. J Am Coll Nutr 1993;12:26-30]. To minimize this interaction, individuals should take zinc supplements at least 2 hours before or after taking penicillamine.

### Diuretics

Thiazide diuretics such as chlorthalidone (Hygroton®) and hydrochlorothiazide (Esidrix® and HydroDIURIL®) increase urinary zinc excretion by as much as 60% [Wester P O. Urinary zinc excretion during treatment with different diuretics. Acta Med Scand 1980;208:209-12]. Prolonged use of thiazide diuretics can deplete zinc tissue levels; and accordingly, clinicians routinely monitor zinc status in patients taking these medications.

### E. Zinc Containing Compositions as Topical Anti-Skin Cancer Agents

### Historical Origins of the Concept

Dr. Frederic E. Mohs was the originator of the idea for using zinc containing compositions in admixture with other substances topically as an anti-skin cancer agent. During the 1930's, Dr. Mohs observed that the injection of zinc chloride into cancerous tissue not only caused tissue necrosis (cell death), but also noted that the microscopic structure of the killed tissue was retained as if the tissue had been surgically excised and immersed in a histologically preserving solution. Then in 1941, Dr. Mohs wrote multiple medical articles and published a textbook on the successful surgical excision treatment of skin cancer and melanoma using an anti-skin cancer paste containing zinc chloride and the escharotic bloodroot plant, Sanguinaria canadensis. The specific formulation of Dr. Mohs' anti-skin cancer paste was: Stibnite (alpha, beta-Diphenylethylene 80-mesh sieve), 40 g; Sanguinaria canadensis, 10 g; and zinc chloride, saturated solution, 34.5 mL (zinc chloride 45% by weight).

Dr. Mohs identified his paste as “zinc chloride fixative paste”, and referred to the skin excision surgery as “chemosurgery” or “fixed-tissue micrographic surgery”. Procedurally, Dr. Mohs employed a layer by layer surgical excision technique in combination with the in-situ fixation of the tumorous tissue with zinc chloride fixative paste for the treatment of skin melanoma. The Mohs zinc chloride fixative paste and his surgical excision procedure is described in full by U.S. Pat. No. 2,344,830, the text of which is expressly incorporated by reference herein.

In brief, the Mohs' fixed-tissue skin excision surgery is performed as follows: A clinically apparent melanoma is first treated with zinc chloride fixative paste prior to any biopsy or debulking procedure. The next day, a layer of the fixed skin tissue is surgically excised, and frozen histologic sections are then made for microscopic examination to confirm the clinical diagnosis of melanoma. The melanoma is then surgically excised layer-by-layer—with each successive layer of tissue first fixed in-situ, and then conservatively removed (for subsequent microscopic scanning of the entire undersurface utilizing frozen histologic sections cut horizontally from the bottom of the excised specimens). The zinc chloride fixative paste is reapplied as necessary until a melanoma-free plane of skin tissue was reached.

### More Recent Developments

A number of more recent developments have been made which are directed to topical uses of zinc containing compositions as skin treatment agents. Merely representing and illustrating these newer zinc containing topical preparations are the following:

(i) U.S. Pat. No. 6,558,694 which discloses a unit dose packaging for treating skin cancer, moles, warts, keratoses, skin tumors and melanoma. Each unit dose package consists of: a backing; a zinc chloride mixture consisting of zinc chloride, an alkaloidal herb, and an inert matrix, wherein the zinc chloride mixture is disposed within the inert matrix, and comprises 45% concentration of zinc chloride by weight, an 11% concentration by weight of the alkaloidal herb Sanguinaria canadensis, and at least a 0.001% concentration by weight of the alkaloidal herb taxoid, taxol, paclitaxel, or a derivative thereof; a matrix substrate attached to the backing and embedded with the zinc chloride mixture; and a peel away strip for encapsulating the zinc chloride mixture between the backing and the peel away strip.

(ii) U.S. Pat. No. 4,229,437 which discloses a composition which is a substantially uniform admixture of three parts by volume of bittersweet dried and ground root bark, and one part by volume of zinc chloride (butter of zinc) crystals. This composition is useful for removing an external surface lesion from the skin when the composition is applied in a thin layer directly on the lesion and secured there in an air-tight fashion for a period of time, while keeping the lesion and surrounding area dry and warm. The composition thus has an active component which, when compounded with zinc chloride, forms a medicament paste or salve useful for removing unwanted skin growths.

(iii) U.S. Pat. No. 4,335,110 describes a pharmaceutical composition for the treatment of periodontal and dental diseases, bilharziasis, and skin infections by pseudomonas aeruginosa, cocci and fungi. The composition comprises zinc chloride, the active ingredients of sanguinaria and galangal, and purified water as an excipient. Preferred proportions of the constituents are, by weight: zinc chloride 45%; sanguinaria 10.8%; galangal 7.2%; water 37%. These proportions produce a paste, and an excipient such as petroleum jelly may be added in such a proportion as to produce an ointment. Dilution may be effected by the addition of distilled water or gelatin, and a quantity of the order of 2%, by weight, of sodium carboxy-methyl cellulose may be added as a drying agent.

(iv) U.S. Pat. No. 4,515,779 reveals a composition which comprises principally powdered bloodroot powdered ginger root, and zinc chloride in relatively equal parts by weight. The composition is applied in a number of treatments to skin lesions such as epithelioma tumors. After a short time, the growth comes out and a healing ointment comprising lard, lanolin, phenol and tannic acid powder is applied to the site until healing is effected.

(v) U.S. Pat. No. 4,588,590 discloses an improved method of treating keratosis comprising periodically applying to the affected area a nail polish composition containing an effective amount of at least one corrosive agent which gradually wears away the keratotic lesion by chemical action. A suitable corrosive agent is identified by its corrosive, inflammatory, and irritating action on the affected area of the skin. The corrosive agent must also be non-toxic and otherwise pharmaceutically acceptable. Exemplary corrosive agents include zinc chloride; acids such as ascorbic acid, glacial acetic acid, lactic acid, salicylic acid, trichloroacetic acid; calcium pantothenate; and podophyllum resin.

(vi) U.S. Pat. No. 4,896,727 discloses a method of enhancing penetration of the skin and mucous membrane by a pharmacologically active agent, and the retention of the pharmacologically active agent therein. The method comprises the application to the skin of a composition comprising said pharmacologically active agent and an effective penetrating and retaining amount of a water-soluble zinc-containing compound (which acts to reduce the transdermal flux through the skin and mucous membrane of the pharmacologically active agent). The water-soluble zinc-containing compounds include zinc halide, zinc sulfate, zinc nitrate, zinc acetate, and/or zinc stearate, and most preferably zinc chloride. The pharmacologically active agents with which the water-soluble zinc-containing compounds are used are preferably those containing hydroxyl, oxo, sulfhydryl, amine, carboxyl, and other anionic groups in configurations which readily allow complexation or chelation with zinc ions.

### F. Non-Topical Anti-Cancer Agents Containing Zinc In-Vitro Assays

All in-vitro test systems have inherent limitations in their ability to model whole organism responses; and specific test parameters and endpoints must be identified and appropriately considered when developing in-vitro assays which are predictive of in-vivo efficacy. The development of various in-vitro tests which are indicative of live animal testing are in various stages of evolution; and each of the in-vitro tests used in medical research must be validated as a reliable alternative to the testing of living subjects.

Over the last fifty years, medical research has developed a variety of in-vitro methods intended for use as screens, adjuncts, and replacements for current in vivo test procedures and standards. For example, although technical progress in the development of non-whole animal testing methods has occurred, to date, no single test, or battery of in-vitro tests, has been accepted by the scientific community as a replacement for the animal and human clinical trials currently used for the testing of new drugs. The reason is clear: For replacement of the in vivo standard with in vitro tests, further research is needed to better understand the complex mechanisms of action which exist in vivo for a living subject. Criteria for the validation and acceptance of in vitro methodologies intended to replace in vivo models therefore needs to be well defined; moreover, new risk assessment paradigms to analyze information generated by in vitro methods remain also to be developed.

### The Illusion of False and Unrealistic Presumptions Based on In-Vitro Data

In so far as is known to date, although there are a number of published in-vitro studies reporting that zinc seems to have an effect upon a variety of cancer cell lines maintained in culture media, the in-vivo use of zinc salts as an effective treatment of non-topical cancers in humans has never been successfully performed or reported in the literature. The progress of Zinc compounds into clinical trial for the systemic treatment of non-topical cancer was clouded by physicians' concerns regarding potential Zinc toxicity. The absence of such in-vivo data in scientific publications is not due to a lack of interest, nor to an oversight, nor to a failure of effort. To to a failure of effort. To the contrary, the general absence of in-vivo data demonstrating the efficacy of non-topical anti-cancer compositions and agents which contain zinc is simply that such in-vivo data does not exist.

In this regard, there appears to be a fundamental reason for the general absence in-vivo data which demonstrates the efficacy of non-topical anti-cancer compositions and agents. It has been a long-recognized medical axiom that—compositions which initially appear promising as a new cancer treatment based upon in-vitro data, are commonly and routinely subsequently shown to be a dismal failure when evaluated under in-vivo conditions. This unfortunate reality and fact is most particularly true when the in-vivo test subjects are human patients afflicted with a recognized form of cancer and when the substance being tested is a hoped for new anti-cancer drug. Accordingly, all suggestions and inferences drawn from published research studies concerning new potential compositions and agents for cancer treatment, no matter how impressive the initially reported in-vitro data, must be tempered with the knowledge and disillusionment of many prior failures when a promising new agent was subjected to in-vivo testing. For this reason, all reported in-vitro data must accordingly be considered to be of doubtful efficacy and value until directly evaluated and confirmed by at least some in-vivo, and preferably human, case studies.

### Representative Publications of In-Vitro Research Studies

With the above-identified reservations and concerns about in-vitro research data in mind, attention is directed to the following published studies:

(a) Park K et al, British Journal of Pharmacology, 2002:137:597-607 & Jaiswal A, Narayan S. Journal of Cellular Biochemistry, 2004;92:345-357. These publications reported in-vitro data which demonstrated that zinc chloride inhibits proliferation of human colorectal cancer cells in-vitro.

(b) Iitaka M et al, Journal of Endocrinology, 2001;169:417-424. This published study presented in-vitro data that zinc chloride inhibits proliferation and induces apoptosis in human thyroid cancer cells in vitro.

(c) Liang J et al, Prostate, 1999;40:200-207. This study demonstrated that zinc sulfate and zinc acetate inhibit proliferation and induce apoptosis in human prostate cancer cells.

(d) Shah M et al, Journal of Experimental & Clinical Cancer Research, 2009;28:84-94. This study reported that treatment of prostate cancer cells with zinc acetate at a dose of 100 microM produced cytostatic effects. Specifically, a treatment dosage of 400 microM of zinc acetate induced apoptosis in prostate cancer cells with 50% cell death occuring by 6 hours; whereas with zinc acetate treatment, more than 95% of prostate cancer cells perished within 24 hours.

(e) Ilouz Ret al, BBRC 2002;295:102-106. This study shows that zinc is a potent inhibitor of GSK-3 (Glycogen Synthase Kinase-3), with IC₅₀=15 microM.

(f) Ougolkov A et al, Cancer Research, 2005;65:2076-2081. This study utilized GSK-3 as a new therapeutic target in human cancer.

(g) Ougolkov et al, Clinical Cancer Research, 2006;12:5074-5081; Ougolkov et al, BBRC, 2005;334:1365-1373; Ougolkov et al, Blood, 2007:110:735-742; Bilim V et al, British Journal of Cancer, 2009; in press. All of these studies showed that inhibition of GSK-3 suppresses proliferation and survival of cancer cells in pancreatic cancer, colon cancer chronic lymphocytic leukemia, and kidney cancer.

(h) Cao Q et al, Cell Research, 2006;16:671-677. This study showed that inhibition of GSK-3 suppresses proliferation and survival of ovarian cancer cells.

(i) Kunnimalaiyaan Met al, Mol Cancer Therapy, 2007;6:1151-1158. This study showed that inhibition of GSK-3 suppresses proliferation and survival of thyroid cancer cells.

(j) Sun A et al, Prostate 2007;67:976-988. This study showed that inhibition of GSK-3 suppresses proliferation and survival of prostate cancer cells.

(k) Kotliarova S et al, Cancer Research, 2008;68:6643-6651. This study showed that inhibition of GSK-3 suppresses proliferation and survival of glioma cancer cells.

(l) Wang Z et al, Nature, 2008;455:1205-1209. This study showed that inhibition of GSK-3 suppresses proliferation and survival of mixed-lineage leukemia cancer cells.

### Other Publications Identifying Zinc Containing Substances as Possible Non-Topical Anti-Cancer Agents

(1) U.S. Pat. No. 7,528,125: This patent discloses chemically sophisticated zinc ionophores, zinc chelators and zinc complexes having an enhanced aqueous solubility for potential use as transport molecules in the treatment of cancer cells. As described, the zinc ionophores are water-solubilized zinc (1-hydroxypyridine-2-thione) or “ZnHPT” compounds—i.e., chemical analogues of pyrithione (which is insoluble in water, is poorly bioavailable, and is thus deemed to be unsuitable for medical purposes). These water-solubilized zinc ionophores are said to be capable of transporting zinc ions between the extracellular environment and the intracellular environment. Similarly, the zinc chelators are multi-dentate ligands that can coordinate to a zinc ion to form a zinc complex; and the zinc complexes comprise at least one zinc ion chelated by at least one multi-dentate ligand.

These zinc ionophores, zinc chelators, and zinc complexes having an enhanced aqueous solubility are said to have increased solubility and bioavailability upon administration when compared to other zinc pyrithione analogues. It is believed that such water-solubilized analogues of pyrithone might be employed as transporting molecules which can bind and carry zinc into cells; and, via such transport, thereby increase intracellular zinc levels. However, the ability of these structurally sophisticated, water solubilized carrier molecules to deliver zinc into cancer cells is demonstrated solely by in-vitro test data and using a tumor xenograft mouse model.

(2) WIPO Publication WO 2009/039508 (published Mar. 26, 2009): This WIPO publication discloses the response of normal human cells maintained in culture to ZnO nanoparticles under different signaling environments, and compares it to the response of cancerous cells maintained in culture. The data reveals that the introduced ZnO nanoparticles exhibited a strong preferential ability to kill cancerous T cells (about 0.28×-35×) in comparison to normal cells.

As described, the activation state of the cell contributes toward ZnO nanoparticle toxicity—i.e., resting T cells displayed a relative resistance, while cells stimulated through the T cell receptor and CD28 costimulatory pathway show greater toxicity in direct relation to the level of activation. The findings of cell selective toxicity for potential disease causing cells indicate a potential utility of ZnO nanoparticle for the treatment of cancer and/or autoimmunity; and the innovation is defined as a method for preferentially killing cancer cells relative to normal cells by treating the cells with zinc oxide nanoparticles.

(3) WIPO Publication WO/2009/072120 (published Jun. 11, 2009): Although legally not “prior art” as such, this WIPO publication discloses that Zn²⁺ concentration is low in HIPK2i cells maintained in culture; is carefully regulated by cellular metalloproteins in HIPK2i cells; and that the actual concentration of free Zn in HIPK2i cells is not known. The text describes the treatment of the misfolded inactive p53 protein in HIPK2i cells, where zinc was added to the culture medium concomitant with the anti-cancer drug adriamycine (ADR). Surprisingly, zinc supplementation restored wtp53 transcription activity, as demonstrated by in-vitro assays. In addition, immunoprecipitation with conformation-specific antibodies showed a marked increase in p53 reactivity to PAbI 620 (the folded/wild-type conformation).

This observation together with the effect of antibody 1801 suggested that a unique region within the N-terminus encompassing Ser46 may influence the folding of the DNA binding domain of p53 protein. Moreover, by ChIP analysis, it was shown that the promoters occupancy by p53 shifted from the non canonical promoter (MT2A, “oncogenic” function) to canonical p53 target gene promoters (DR5, BTG2, p53R2, oncosuppressor function) following the combined ADR and zinc treatment. It is then hypothesized that zinc may facilitate p53 binding to the target site in vivo, as it does in vitro, through regain of wild-type conformation state—although the precise mechanism remains to be elucidated.

The effect of the anti-cancer drug adriamycine (ADR) in combination with zinc was also tested in-vivo on tumor-bearing mice. Mice bearing tumors were treated with a combination of ADR and zinc; and this combination resulted in a significant synergistic inhibition of tumor growth. The effect on the HIPK2-deficient tumors is attributed to the regain of wtp53, as was shown in-vitro assays; and implies restoration of the tumor response to adriamycine treatment, although p53-independent HIPK2 suppressor functions cannot be completely ruled out. The neoplastic potential due to reduction of HIPK2 function has been ascribed to impairment of p53 oncosuppressor functions. The reported data suggest a model in which loss of (p)Ser46 by HIPK2 leads to misfolded p53 state that allows some gain-of-function and a switch from oncosuppressor to oncogene function. It is however significant that this “gain of function” attributed usually to mutant p53 is exercised by wtp53; and thus wtp53 is said to show conformation dependent tumorigenicity.

(4) Publication KR/2003/080867 (published Oct. 17, 2003): This publication discloses that a zinc compound has growth inhibitory activity of large intestinal cancer cells which were maintained in culture through expression of p21Cip/WAF1 and transport thereof to a nucleus. In particular, the publication states that the activation of the p21Cip/WAF1 is achieved through strong and long-lasting activation of the signal transduction system of ERK MEK-2A. The procedure uses a zinc compound; exhibits a correlation between an inhibitory and controlling mechanism of human large intestinal cancer cells; and demonstrates the activation of an ERKs pathway by zinc.

### SUMMARY OF THE INVENTION

The present invention has multiple aspects for therapeutically treating human cancers in-vivo.

A first aspect provides an oral method for therapeutically treating a living human subject afflicted with a recognized type of cancer, said oral therapeutic treatment method comprising the steps of:

obtaining an orally ingestible preparation comprising a zinc compound selected from the group consisting of simple inorganic salts of zinc and simple organic salts of zinc and a biocompatible carrier composition suitable for oral ingestion;

orally administering not less than about 200 milligrams of said zinc compound preparation as an anti-cancer medicament to the afflicted human subject over 24 hours time;

repeating said oral administration of said zinc compound preparation as an anti-cancer medicament to the afflicted human subject every day as a therapeutic regimen for at least an initially scheduled treatment time;

periodically monitoring the status of the afflicted human subject during said oral treatment regimen to confirm that manifestations of zinc toxicity have not appeared; and

clinically determining that said oral treatment regimen using said zinc compound preparation as an anti-cancer medicament has been effective and that the recognized type of cancer at least has become demonstrably stabilized in the afflicted human subject.

A second aspect of the invention presents a parenteral method for therapeutically treating a living human subject afflicted with a recognized type of cancer, said parenteral therapeutic treatment method comprising the steps of:

obtaining a sterile preparation comprising a zinc compound selected from the group consisting of simple inorganic salts of zinc and simple organic salts of zinc and an aqueous carrier fluid suitable for parenteral administration;

parenterally administering not less than about 0.5 milligrams per kilogram of body weight of said zinc compound preparation as an anti-cancer medicament to the afflicted human subject over 24 hours time;

repeating said parenteral administration of said zinc compound preparation as an anti-cancer medicament to the afflicted human subject every day as a therapeutic regimen for at least an initially scheduled treatment time;

periodically monitoring the status of the afflicted human subject during said parenteral treatment regimen to confirm that manifestations of zinc toxicity have not appeared; and

clinically determining that said parenteral treatment regimen using said zinc compound preparation as an anti-cancer medicament has been effective and that the recognized type of cancer at least has become demonstrably stabilized in the afflicted human subject.

This parenteral treatment method includes the intra-arterial, intravenous, intraperitoneal, intramuscular, intradermal, subcutaneous and intratumoral modes of administration.

A third aspect of the invention offers a body cavity method of therapeutically treating a living human subject afflicted with a recognized type of cancer, said body cavity therapeutic treatment method comprising the steps of:

obtaining a body cavity acceptable preparation comprising a zinc compound selected from the group consisting of simple inorganic salts of zinc and simple organic salts of zinc and a biocompatible carrier suitable for administration to a body cavity in an afflicted human subject;

administering not less than about 0.5 milligrams per kilogram of body weight of said zinc compound preparation as an anti-cancer medicament to a body cavity of the afflicted human subject over 24 hours time;

repeating said body cavity administration of said zinc compound preparation as an anti-cancer medicament to the afflicted human subject every day as a therapeutic regimen for at least an initially scheduled treatment time;

periodically monitoring the status of the afflicted human subject during said body cavity treatment regimen to confirm that manifestations of zinc toxicity have not appeared; and

clinically determining that said body cavity treatment regimen using said zinc compound preparation has been effective and that the recognized type of cancer at least has become demonstrably stabilized in the afflicted human subject.

This body cavity treatment method includes the administration of drug specifically into the abdominal, thoracic, rectal, or vaginal cavities.

### DETAILED DESCRIPTION OF THE PRESENT INVENTION

The present invention is a novel method of treating different kinds of cancer in humans using at least one simple organic or inorganic zinc salt as an anti-cancer medicament. The methodology as a whole provides for a non-topical administration in high dosage of one or more simple zinc salts to the afflicted human cancer patient every day for at least an initially scheduled treatment time; and utilizes a non-topical manner of introduction—i.e., an oral, parenteral, and/or body cavity mode of administration—to deliver at least a minimally effective concentration of a simple zinc salt to the cancer cells then present within the organs and tissues of the living human subject.

### I. The Unique Aspects and Unforeseen Effects of the Present Invention

The present methodology is a unpredicted approach and unexpected medical regimen which is demonstrably effective in-vivo for the therapeutic treatment of various recognized forms of cancer in humans. Among the singular requirements and unique results which markedly separate and distinguish the present invention from its predecessor treatment methods are the following:

1. The present method employs surprisingly large concentrations and greater than normal dosages of at least one zinc containing compound as a daily therapeutic treatment. Thus, when administered either orally or via an externally located body cavity to the human patient, the minimal effective concentration and dosage of the zinc containing compound will not be less than 200 milligrams (mg) daily; and when administered parenterally, the minimal effective concentration and dosage of the zinc containing compound will be at least 0.5 milligrams per kilogram (mg/kg) of patient body weight over 24 hours time.

It will be appreciated also that in this methodology, when administered either orally or via an externally located body cavity to the human patient, the maximal effective concentration and dosage of the zinc containing compound is about 2000 milligrams (mg) daily; and when administered parenterally, the maximal effective concentration and dosage of the zinc containing compound is about 10.0 milligrams per kilogram (mg/kg) of patient body weight over 24 hours time. No conventionally known mode of medical treatment uses, or would even consider administering, such unusually high dosages of a zinc containing compound every 24 hours to any human cancer patient.

2. The present methodology stands in direct opposition to and in contradiction of the overt teachings and direct warnings regarding zinc toxicity in humans as disclosed in the relevant prior art publications. Nevertheless, the unusually large concentrations and greater than normal dosages of a zinc containing compound administered to the cancer patient as a daily therapeutic treatment demonstrably do not initiate nor cause clinical manifestations of zinc toxicity in the afflicted cancer patients (Clinical Case Histories 1-3).

This clinical outcome and unexpected effect is not merely verbiage nor hyperbole. To the contrary, direct proof and factual evidence of this extraordinary phenomenon is provided by the clinical treatments and individual case histories of human cancer patients presented hereinafter. Moreover, in so far as is known to date, this particular aspect and clinical result—the absence of clinical manifestations indicating zinc toxicity in human cancer patients actually receiving unusually large concentrations and greater than normal dosages of a zinc containing compound as a daily treatment—is unknown and unfeasible given the conventional teachings and beliefs in the published medical literature.

3. The present methodology can cause two identifiable kinds of therapeutic effects in-vivo: In a first instance when merely a minimally effective concentration of a zinc containing compound is administered daily to the cancer patient, the minimalist treatment regimen is able to retard primary or metastatic tumor growth; and stop cancer progression, leading to stabilization of the disease in-vivo. In comparison, in a second preferred instance when more than a minimally effective concentration is administered daily to the cancer patient, the preferred treatment regimen will cause a true regression of the cancer over time in the afflicted patient. These separate and distinct in-vivo capabilities are demonstrated by the individual case histories of human cancer patients presented hereinafter.

4. The present invention does not depend upon the water solubility characteristics of the chosen zinc containing compound for therapeutic efficacy in-vivo. Unlike the strictured views and conventional orientation of the prior in-vitro art concerning the potential use of water soluble zinc which is more convenient to use in in-vitro experiments as an anti-cancer agent, the present invention does not insist nor rely on a single mechanism of action; and particularly does not depend upon one which is based entirely upon the capability of the zinc containing substance to dissolve in water and concomitantly release free zinc ions into an aqueous based fluid.

Instead, the instant methodology intends and expects that multiple and different mechanisms of action will be concurrently active in-vivo; and that these different mechanisms will be individually and collectively in effect as a consequence of administering a simple organic or inorganic salt of zinc orally, parenterally, and/or via an externally exposed body cavity to a cancer patient. These multiple mechanisms of action each provide in-vivo activity and therapeutic efficacy for a variety of different zinc containing compounds that are well known to be either only slightly soluble, or are hardly soluble, or are substantially insoluble in water and aqueous based fluids.

The existence of such different mechanisms of action in-vivo within cancer patients is amply demonstrated and evidenced via several human case histories which employed zinc picolinate (which is only slightly soluble in water and aqueous based fluids) as the chosen simple organic salt of zinc for therapeutic treatment purposes, as is empirically demonstrated in-vivo by clinical case histories nos. 1-3 respectively presented hereinafter. Yet, the use of simple zinc salts which are either only slightly soluble, or are hardly soluble, or are substantially insoluble in water and aqueous based fluids as efficacious anti-cancer agents is unknown and contrary to the expectations of the ordinary medical researcher and clinical practitioner working in this field.

### II. Terminology, Jargon, and Definitions

Although many of the words, terms and titles employed herein are commonly employed and conventionally understood in their traditional usage and context by persons ordinarily skilled in this art, a short listing of definitions is presented below in order to provide a minimal vocabulary; and as an aid and guide for avoiding misinformation, misunderstandings, and ambiguities in terminology which often exist in this technical field; and to introduce specialized terms and jargon for recognizing the particulars of the present invention and for appreciating the true scope and breadth of the claims recited below.

Cancer: A term that, by common usage, has come to encompass any and all forms of malignant neoplasms.

Tumor: A swelling as such; but which has, by common usage, become a substitute term for ‘cancer”, but properly includes benign neoplasms as well.

Anti-cancer medicament, agent, or drug: Any substance, formulation, composition, or preparation used for medical treatment of a living human subject afflicted with a recognized form of cancer.

Salt: A compound which results from the replacement of one or more hydrogen atoms of an acid by metal atoms or electropositive radicals, and forms positive and negative ions upon dissolution.

Simple inorganic salt of zinc: A compound which is devoid of carbon and results from positively charged zinc ions combining with a negatively charged non-metallic ion.

Simple organic salt of zinc: A carbon containing compound which is neither complicated nor elaborate in formulation and results from a negatively charged carbon containing radical combining with a positively charged zinc ion.

Minimal effective concentration of a zinc containing compound: The smallest useful dosage of a zinc containing compound which is able to cause a suppression of tumor growth and cancer progression in-vivo for a human patient.

Preferred effective concentration of a zinc containing compound: A generally desirable dosage range of a zinc containing compound which is not only able to able to cause a suppression of tumor growth and cancer progression in-vivo but also is able to induce a regression of primary and/or metastatic cancer in human patient.

Maximum effective concentration of a zinc containing compound: The largest useful dosage of a zinc containing compound which is able to induce a regression of primary and/or metastatic cancer in human patient.

### III. The Range and Variety of In-Vivo Treatable Human Cancers

A neoplasm is an abnormal mass of cells typically exhibiting uncontrolled and progressive growth in-vivo. Neoplasms are broadly classified into two categories: (1) benign and (2) malignant.

Benign neoplasms are non-invasive; are frequently encapsulated; and do not metastasize. Typically, but not invariably, these are slow growing masses of cells; and though they remain localized, benign neoplasms occasionally cause death from complications such as compression of a critical structure or hemorrhage.

In contrast, malignant neoplasms generally grow more rapidly that benign neoplasms, can invade adjacent tissues, and metastasize. Malignant neoplasms are classified into two general categories: (1) malignant tumors of epithelial origin (e.g. carcinomas) and (2) malignant tumors of mesenchymal origin (e.g. sarcomas).

Note also that a pre-invasive stage of a malignant epithelial tumor is medically recognized and designated as “in-situ” or intraepithelial carcinoma. When an invasion of cancer cells to adjacent normal tissues and structures does occur, the access of malignant cells to lymph and blood vascular channels provides the means for dissemination of cancer cells (metastasis) to distant parts of the human body. The newly established metastatic foci become centers for further growth as satellite tumors. Metastases are frequently the cause of death of the human host, although either the primary tumor or a metastasis may interfere with the functions of vital organs.

### The Range and Variety of Human Cancers Treatable Using the Present Methodology

The therapeutic methods of the present invention are suitable and effective for treatment of a diverse range of recognized human cancers occurring in-vivo. Merely illustrating and exemplifying the intended range of treatable human cancers are those listed by Table 1 below:

TABLE 1
Treatable Human Cancers

Class A: cancer of oral cavity, salivary gland cancer, esophageal
cancer, gallbladder cancer, cancer of bile ducts, liver cancer, stomach
cancer, pancreatic cancer, colon cancer, rectal cancer, anal cancer,
gastrointestinal stromal tumors, gastrointestinal carcinoid tumors.
Class B: breast cancer, cervical cancer, endometrial cancer, ovarian
cancer, and vaginal cancer.
Class C: bladder cancer, testicular cancer, prostate cancer, kidney
cancer (renal cell carcinoma), adrenal cancer, and penile cancer.
Class D: head and neck cancer, nasal cancer, laryngeal cancer,
thyroid cancer, eye cancer, retinoblastoma, skin cancer, basal cell
carcinoma, melanoma, and Kaposhi's sarcoma.
Class E: lung cancer, malignant mesothelioma.
Class F: aplastic anemia, Non-Hodgkin's lymphoma, Hodgkin's
lymphoma, acute lymphocytic (lymphoblastic) leukemia, T-cell leukemia,
acute myeloid leukemia, chronic lymphocytic leukemia, chronic myeloid
leukemia, multiple myeloma, myelodysplastic syndrome, hairy cell
leukemia, and lymphoplasmacytic lymphoma (Waldenstrom's
macroglobulinemia).
Class G: tumors of central nervous system (CNS), astrocytoma,
medulloblastoma, CNS lymphoma, and pituitary tumor.
Class H: bone cancer, osteosarcoma, rhabdomyosarcoma, sarcoma,
metastatic tumors, abdominal cavity metastasis, bone metastasis, liver
metastasis, lung metastasis, and brain metastasis.

### IV. The Simple Organic and Inorganic Salts of Zinc Employed as Efficacious Anti-Cancer Medicaments

The present methodology for treating human cancers in-vivo comprises the administering of one or more chosen simple organic or inorganic salts of zinc, at not less than a minimally effective concentration, to a living human cancer patient as a daily therapeutic regimen. Merely illustrating and exemplifying the range of available simple organic or inorganic salts of zinc which are used as anti-cancer medicaments are those representative compounds listed by Table 2 below.

TABLE 2
Simple Organic And Inorganic Salts Of Zinc

| Organic Salts Of Zinc | Molecular Formula | Water Solubility |
|---|---|---|
| Zinc Picolinate | C₁₂H₈N₂O₄Zn | slightly soluble |
| Zinc Gluconate | C₁₂H₂₂O₁₄Zn | soluble |
| Zinc Acetate | C₄H₁₀O₆Zn | highly soluble |
| Zinc Glycinate | C₄H₈N₂O₄Zn | slightly soluble |
| Zinc Methionine Sulfate | C₅H₁₁NO₆S₂Zn | soluble |
| Zinc Monomethionine | C₅H₁₂NO₆S₂Zn | soluble |
| Zinc Pyrithione | C₁₀H₈N₂O₂S₂•Zn | insoluble |
| Zinc Acexamate | C₁₆H₂₈N₂O₆Zn | soluble |
| Zinc Ascorbate | C₁₂H₁₄O₁₂Zn | soluble |
| Zinc Aspartate | C₈H₁₂N₂O₈Zn | soluble |
| Zinc Citrate | Zn₃C₁₂H₁₀O₁₄ | insoluble |
| Zinc Orotate | C₁₀H₆N₄O₃Zn2H₂O | hardly soluble |
| Zinc Glycerate | C₆H₁₀ZnO₈ | soluble |
| Zinc Propionate | C₆H₁₀O₄Zn | highly soluble |
| Zinc Lactate | C₆H₁₀O₆Zn | highly soluble |
| Zinc Sulfate | ZnSO₄ | highly soluble |
| Zinc Chloride | ZnCl₂ | highly soluble |
| Zinc Carbonate | ZnCO₃ | insoluble |

### The Range of Therapeutic Outcomes Effected In-Vivo by the Simple Zinc Salts

It will be noted and appreciated that the present methods expect and intend that not less than two different and distinct therapeutic outcomes can be obtained in-vivo. Thus, if only a minimally effective concentration of the prechosen simple zinc salt is administered, a suppression of tumor growth will occur in-vivo over time for the cancer-afflicted human patient and the tumor becomes stabilized within the patient.

In contrast, if a preferred, or optimal effective concentration of a prechosen simple zinc salt is administered, this greater than minimal dosage will generally not only be able to cause a suppression of tumor growth in-vivo, but also will be able to induce a regression of tumor in-vivo for the cancer-afflicted human patient.

Ultimately, if a maximum effective concentration of a zinc containing compound is administered, this maximal dosage of a zinc containing compound will not only be able to initiate and directly cause a prolonged regression of tumor in-vivo, but also in many instances cause a clinically verifiable elimination of the tumor for the afflicted human patient.

### V. Therapeutically Efficacious Preparations of Simple Zinc Salts

The zinc containing compounds listed by Table 2 above can be administered in either single or multiple doses by any of the accepted modes of administration including oral, parenteral, and body cavity routings. Given the choice of using any of these different modes of administration, then the preparation of the chosen simple organic or inorganic salt of zinc will conform to the particular requirements demanded by that specific mode of administration. Clearly, any complete formulation of the anti-cancer medicament will provide not less than a minimally effective concentration of the chosen zinc containing compound in combination with a biocompatible carrier which is suitable for and acceptable with the intended mode of administration.

### For Oral Administrations

### Efficacious Dosages for Oral Administration

A most preferred mode of administration is by oral ingestion. To treat cancer afflicted human patients, an effective concentration of zinc for oral intake will vary from about 200 mg (the minimal effective concentration) to not more than 2000 mg (the maximal effective concentration) per day. A preferred effective concentration for oral ingestion will range from about 600 mg to about 1,000 over 24 hours time.

Although the entire prescribed oral dosage of zinc can be given to the patient as a single daily administration, it is far more preferable that the prescribed total daily dosage of zinc be given only in part as a series of 2-5 individual administrations at fixed time intervals over each 24 hour time period. In this manner, a more uniform level of zinc containing compounds are present within the human body at any given time; and a more consistent therapeutic effect is maintained over each 24 hour treatment regimen.

### Acceptable Biocompatible Carrier Formulations for Oral Administration

It is expected and intended that the formulations of the complete oral preparation will be made in either liquid or solid formats; and will exist in a dosage range such that the desired total daily dose can be orally ingested in whole or in parts over each 24 hour time period by the patient.

Thus, in the liquid formats, an aqueous based fluid can be prepared which will typically include not only water—but also a limited quantity of sweetening agents, flavoring compositions, and antimicrobial compounds or preservatives to make a palatable liquid which can be easily swallowed by the patient on a scheduled regimen. In addition, if and when desired, a conventionally known range of thickening agents and/or gelling agents may be usefully added to the preparation to make a flowing syrup-like product.

Alternatively, in the solid formats, the chosen simple zinc salt will be prepared in powdered or pulverized form as either a tablet or capsule which can be swallowed every 4-6 hours by the cancer patient in accordance with a fixed treatment regimen, and will provide the desired total daily dosage of zinc every 24 hours. These tablets or capsules containing the desired dosage are conventional pharmaceutical preparations; and combine the chosen zinc containing compound with a variety of biocompatible substances which typically include starch, caffeine, acid buffering agents, and blending agents.

### For Parenteral Administrations

### The Alternative Modes of Parenteral Administration

Parenteral administrations generally include the intra-arterial, intravenous, intraperitoneal, intratumoral, intramuscular, intradermal, and subcutaneous routings and modes. In typical medical practice, parenteral administrations are performed by infusion or by injection of a properly prepared fluid containing a chosen simple zinc salt.

### Efficacious Dosages for Parenteral Administration

To treat cancer afflicted human patients, an effective concentration of at least one simple zinc salt for parenteral administration (via infusion or injection) will vary from about 0.5 mg/kg of patient body weight (the minimal effective concentration) to not more than about 10.0 mg/kg of patient body weight (the maximal effective concentration) over 24 hours time. However, a preferred effective concentration for parenteral treatments will range from about 2.0 to about 8.0 mg/kg of patient body weight over 24 hours time; and an optimal effective concentration for parenteral treatments is believed to be from about 4.0 to about 6.0 mg/kg of patient body weight over 24 hours time.

Although the entire prescribed parenteral dosage of simple zinc salt can be given to the patient as a single daily infusion or injection, it is far more preferable that the prescribed total daily dosage of zinc be administered in parts as multiple parenteral treatments given at fixed time intervals over each 24 hour time period. In this manner, a more uniform level of zinc containing compounds are present in the human body at any given time, and a more consistent therapeutic effect is maintained over each 24 hour treatment period.

Accordingly, to treat cancer patients in this manner, an effective concentration of the chosen simple zinc salt for parenteral administration will be an infusion or an injection which is given as a series of as 2-5 infusions/injections within 24 hours' time in which the total daily dosage administered will vary from as little as 0.5 mg/kg to as much as 10.0 mg/kg of patient body weight.

Note than infusion as such involves the steeping of a substance in water to obtain its soluble principles; and, by definition, is the therapeutic introduction by gravity of a fluid other than blood (such as saline) into a vein. In contrast, injection by definition is the introduction of a fluid (typically a solution containing a medicinal substance) into a body part, or into the vascular system, or into an organ by means of a syringe or other device connected to a hollow needle. The particular routing for infusion or injection thus can be any of the following: the intra-arterial, intravenous, intraperitoneal, intratumoral, intramuscular, intradermal, or subcutaneous routings.

### Acceptable Biocompatible Carrier Formulations for Parenteral Administration

Since the desired effective concentration of the chosen simple zinc salt will be introduced to the human body as an intrusive application by infusion or injection, the appropriate parenteral dosage should be prepared in sterile form; can be made in single or multiple dose formats; and will typically be dispersed in a sterile fluid carrier such as physiological saline, 5% dextrose solutions, serum, or plasma.

It is most desirable that the fluid carrier be biocompatible with the cells and tissues of the human body. For this reason, physiological strength electrolytes are typically present in the carrier fluid. In addition, a broad range of other chemical agents and additives may be included in small quantities as non-essential ingredients in the various formulations of the fluid carrier. These non-essential ingredients can be water soluble, water miscible, or aqueous suspensions; and will typically include various minerals, lidocaine or other local anesthetics, one or more coloring agents, water soluble antimicrobial preservatives, one or more blending agents, and the like.

In addition, various embodiments of the complete parenteral preparation may comprise one or more conventionally known surfactants. The surfactant may be an anionic surfactant, a cationic surfactant, an ampholytic surfactant, or a nonionic surfactant. Suitable concentrations of surfactant typically are between about 0.05% and 2%.

### For Body Cavity Administrations

Generally, almost all cancers occur within human body cavities. For cancer therapy, administration of the medicament to a naturally existing body cavity is made in order to deliver the medicament into the patient's blood circulation or to achieve a local anticancer effect of the drug applied to the tumor.

Anatomically, most body cavities are internalized spaces of varying volume, the largest of these being exemplified by the thoracic and abdominal cavities; as such, these internalized body cavities are suitable for treatment by parenteral administration. Several anatomic body cavities, however, lie externally exposed to and are accessible via the ambient environment. Among these externally exposed body cavities are the rectum, the vagina, and the urethra.

Of these externally exposed body cavities, urethral cancer is a rare event, and urethral administration of a medicament for cancer treatment is quite rarely performed as well.

Note also that both abdominal and rectal administrations are an excellent way to deliver a zinc containing medicament into the bloodstream. In comparison, although a vaginal administration of the medicament may have a minor general effect, it causes primarily a localized anticancer result.

In addition, although bladder cancers can sometimes be treated by an infusion of a medicament through the urethra, this protocol is today a rare manner of treatment. Far more frequently, a medicament will be infused into the abdominal cavity in case of peritoneal metastasis either for local treatment or for delivery into the blood stream, because medicaments administered in this manner are absorbed very well in the abdominal cavity.

### Efficacious Dosages for Body Cavity Administration

An effective concentration of zinc compound introduced via body cavity administration will vary from not less than about 200 mg (the minimal effective concentration) to not more than 2000 mg (the maximal effective concentration) per day. A preferred effective concentration for body cavity administration will typically range from about 600 mg to about 1,000 mg over 24 hours time.

Although the entire prescribed dosage of zinc can be applied to the body cavity of the patient as a single daily treatment, it is far more preferable that the prescribed total daily dosage of the chosen simple zinc salt be given in parts as a series of multiple administrations, at fixed time intervals, over each 24 hour time period. In this manner, a more uniform level of zinc containing compounds will be present in the human body at any moment in time; and a more consistent therapeutic effect is maintained over each 24 hour treatment regimen.

### Acceptable Biocompatible Carrier Formulations for Body Cavity Administration

Owing to the fact that cancers anatomically occurring within the externally exposed body cavities of the human body (e.g., the rectum and the vagina) are generally unsuitable for medicaments which exist in dry, powdered, or liquid states, most suitable pharmaceutical formulations comprising the zinc containing medicament will typically appear in the form of creams, ointments, and suppositories.

Pharmaceutically, a cream is a fluid oil-in-water emulsion, a semisolid dosage format which is either an emulsion of oil and water or an aqueous microcrystalline dispersion of a long-chain fatty acid or alcohol.

In comparison, ointments are fluid water-in-oil emulsions. Although the useful carrier base formulations will vary in their ingredients, ointments generally are relatively thick preparations which employ various hydrocarbon substances (such as petrolatum or different mixtures thereof) with mineral oil as a biocompatible carrier. Often an ointment becomes somewhat more fluid as the temperature increases; and conventional ointment carrier formulations provide some lubrication at the anatomic use site, as well as provide a release of the active ingredients at that anatomic site.

As an alternative, some ointment formulations may employ non-hydrocarbon materials such as polyethylene glycol, or mixtures thereof; and these alternative materials typically provide a more water-soluble ointment base preparation. However, the majority of such non-hydrocarbon ointments have not found general acceptance to date.

Suppositories are also water-in-oil emulsions, but exist primarily as solid dosage forms of a medicament intended for administration within an externally exposed body cavity (such as the rectum or vagina). Suppositories are useful solid drug delivery systems in those situations where it is deemed to be difficult or ineffective to deliver medicine orally, intravenously, or by injection; and such an alternate route of effective administration becomes necessary and appropriate.

Suppositories are formulated using a mixture of different hydrocarbons (such as petrolatum, mineral oils, fatty acids, triglycerides, fats and lipids) to form a biocompatible solid carrier base; and is compounded such that the shaped solid mass will melt, soften, or dissolve within the body cavity where the temperature is around 98.6 degrees F., thereby releasing the active medicament contained therein. Suppository carrier base formulations are stable, nonirritating, chemically neutral, and physiologically inert; and typically utilize a variety of different oily or fatty materials (such as cocoa butter, coconut oil, palm kernel oil, and palm oil) which melt or deform at room temperature. Biocompatible carrier base formulations frequently also contain various alcohols, surfactants, emulsifiers, and other blending agents as additives.

### VI. The Manipulative Steps Comprising the Therapeutic Method

The present invention provides a non-topical method for therapeutically treating a living human subject afflicted with a recognized type of cancer. The individual steps of the therapeutic treatment method include the following manipulative acts:

Step 1: Obtaining a preparation comprising at least one zinc compound selected from the group consisting of simple inorganic salts of zinc and simple organic salts of zinc and a biocompatible carrier suitable for use with the intended manner of administration.

Although the present methodology is focused upon the use of single simple zinc salts and suitable biocompatible carriers, it is envisioned that many clinical treatments will employ two or more different zinc containing compounds concurrently (i.e., simultaneously or in serial sequence) as multiple anti-cancer agents. The use of multiple simple zinc salts as medicaments allows the oncologist or physician not only to combine organic and inorganic compounds; but also to form mixtures of diverse simple zinc salts having markedly different physical and chemical properties. An example of such concurrent use is demonstrated in-vivo by clinical case history no. 2 hereinafter.

For similar reasons, the individual physical and chemical properties of the different biocompatible carriers available for use with each mode of administration (as described above) allows the pharmaceutical manufacturer or physician to pick and choose from among the entire range of simple zinc salts those particular which are deemed to be most preferable with aqueous-based carrier formulations and those other choices most desirable for use with oil-based carrier formulations.

Step 2: Administering (via an oral, parenteral or body cavity routing) a total daily dosage which is not less than a minimally effective concentration and not more than a maximally effective concentration of at least one zinc compound preparation as an anti-cancer medicament to the afflicted human subject over 24 hours time.

The total daily dosage of the simple zinc salt to be administered to any living human patient will of course vary with and be dependent upon the age, overall health, and weight of the recipient; the kind of concurrent treatment, if any; the frequency of concurrent treatment; and the physician's current prognosis for the patient.

It will be noted and appreciated also that the minimal and maximal effective concentration ranges for the oral and the body cavity routes of administration differ from that employed by the parenteral routes of administration. Specifically, the oral and body cavity routes require that an effective concentration of zinc salt is given which will vary from not less than about 200 mg (the minimal effective concentration) to not more than 2000 mg (the maximal effective concentration) per day. A preferred effective concentration for these routes of administration will typically range from about 600 mg to about 1,000 mg over 24 hours time.

In contrast, an effective concentration of simple zinc salt for parenteral administration (via infusion or injection) will vary from about 0.5 mg/kg of patient body weight (the minimal effective concentration) to not more than about 10.0 mg/kg of patient body weight (the maximal effective concentration) per day. Moreover, for parental administrations, a preferred effective concentration for parenteral treatments will range from about 2.0 to about 8.0 mg/kg of patient body weight over 24 hours time; and an optimal effective concentration for parenteral treatments is from about 4.0 to about 6.0 mg/kg of patient body weight over 24 hours time.

The administration of the simple zinc salt via the chosen routing is efficacious over a wide dosage range; and the simple zinc salt is to be administered in a pharmaceutically effective concentration as described above. Nevertheless, the exact amount of simple zinc salt to be administered will be determined by a physician; and will take into account the patient's circumstances, which include cancer type, the chosen route of administration, the age, weight, and response of the patient, the severity of the patient's symptoms, and the like.

Step 3: Repeating the administration of the zinc compound preparation as an anti-cancer medicament to the afflicted human subject daily as a therapeutic regimen for at least an initially scheduled treatment time.

It is expected and intended that the administration of simple zinc salts will performed daily and routinely as a treatment regimen for at least several weeks time. Nevertheless in most instances, the oncologist or physician must expect that the optimum length of time for therapeutic treatment in this manner described herein will likely vary from months to years—depending on the cancer type, the patient's medical condition when beginning treatment, the total daily dosage of zinc salts, the effect of zinc salts on the individual to initiate stabilization of disease, the observed rate for suppressing tumor growth in-vivo, and the speed with which tumor regression can occur in-vivo.

Step 4: Periodically monitoring the status of the afflicted human subject during the therapeutic treatment regimen to confirm that no manifestations of zinc toxicity appear.

It is important to be sure that clinical manifestations of zinc toxicity do not appear in the patient then undergoing therapeutic treatment using the present methodology. The levels of zinc can be monitored in plasma by conventional blood test. A medically normal concentration of zinc in human plasma is about 100 μg/100 mL.

The oncologist or physician should therefore periodically examine his patient in two ways during treatment:

First, the patient should be physically examined on a regular time basis for symptoms of zinc toxicity. As noted above, the risk and dangers of zinc toxicity in humans can occur in both acute and chronic forms; and symptoms of zinc toxicity can appear as nausea, vomiting, loss of appetite, abdominal cramps, diarrhea, and headaches.

Second, samples of the patient's blood should be taken routinely and regularly tested for zinc content using the blood chemistry assays and instrumentation conventionally known and commercially sold for this purpose. Although the values for the individual patient will likely vary considerably, the test results should approximate the medically normal concentration of zinc in human plasma of about 100 μg/100 mL.

In addition, although it is considered very unlikely that any manifestations of zinc toxicity will appear in an individual patient over the duration of therapeutic treatment with the present invention, it is deemed to be proper and necessary to verify that no serious manifestations of zinc toxicity appear regardless of how long the treatment regimen is continued. This is considered to be especially important in view of the likelihood of months and years of therapeutic treatment for some patients. Accordingly, the testing for undesirable zinc effects and toxicity should continue concurrently with and over the full duration of the therapeutic treatment for the patient.

Step 5: Clinically determining that the therapeutic treatment regimen using that dosage of a zinc compound preparation as an anti-cancer medicament has been effective.

In effect, a clinical evaluation and ascertainment is made that the particular type of cancer afflicting the patient at least has become controlled and is demonstrably stabilized in-vivo; and, in the preferred higher dosage instances has regressed, and/or been reduced/eliminated within the afflicted human subject receiving such treatment. Such clinical determinations are typically made by tests using computer tomography, PET-scan, X-ray and endoscopy examinations, blood analyses and evaluations, as well as by other conventionally known diagnostic modalities and clinical test procedures.

### VI. Unusual Features and Unexpected Attributes of the Therapeutic Treatment Method

1. In the present therapeutic treatment methods, the simple organic and inorganic zinc salts alone are the bioactive agents employed as anti-cancer medicaments. Via their in-vivo use in greater than normal dosage concentrations, these simple zinc salts are efficacious compounds which can suppress tumor growth and cancer progression at minimally effective dosages, and cause a regression of tumor at greater than minimally effective concentrations. As such, these zinc containing compounds are intended and expected to be used in the complete absence of any other kind or type of therapeutic agent; and as the sole bioactive agent able to initiate beneficial changes in humans afflicted with a recognized form of cancer, these simple zinc salts constitute the exclusive means of treatment. In short, no other pharmacologically active substance or chemical composition is either needed or desired as an anti-cancer medicament in order to obtain a clinical and demonstrable therapeutic effect in-vivo.

2. If and when thought to be beneficial, and as a purely optional alternative and non-compulsory manner of usage however, one or more simple zinc salts may be combined in admixture and/or concurrently administered with a conventionally known chemotherapeutic drug(s) or agent(s) which are said to be themselves individually effective against human cancers. Nevertheless, even in such optional and alternative use circumstances, it will be recognized and appreciated that the simple zinc salt is and remains the primary and essential therapeutic medicament of choice; and that the predominate role of the simple zinc salt is neither as an enhancer, nor as an adjuvant, nor as an activator or potentiator, nor as a co-factor for any conventionally known chemotherapeutic drug or agent which may then also be present in admixture and/or may be administered concurrently. Accordingly, the absence or presence of any conventionally known chemotherapeutic drug or agent which might concurrently be given to the patient is inconsequential and makes no difference to the efficacy of the present treatment methodology—so long as at least a minimally effective concentration of the chosen simple zinc salt is administered to the cancer patient.

3. Despite the unusually large concentrations and greater than normal dosages of a zinc containing compound being administered to the cancer patient on a regular daily basis, this therapeutic treatment method demonstrably does not initiate, induce, or cause clinical manifestations of zinc toxicity in the afflicted cancer patients. Clear evidence of this remarkable fact and unforeseen outcome is demonstrated by the individual clinical treatments and case histories of human cancer patients presented hereinafter.

This particular clinical result—the absence of clinical manifestations of zinc toxicity in human cancer patients receiving unusually large concentrations and dosages of a zinc containing compound over an extended period of time—is unpredicted and completely unexpected given the teachings and conventional beliefs expressed in the published medical literature. In addition, such an empirically demonstrated absence of toxicity in human cancer patients is a direct contradiction of and stands in clear opposition to what is traditionally presumed to occur under such circumstances by both medical researchers and clinical practitioners alike.

4. It is also important to recognize and appreciate that the present treatment methods can cause two separate and distinct therapeutic effects in-vivo:

(i) If only a minimally effective concentration of a simple zinc salt is administered daily to the human cancer patient, this dosage and treatment regimen will suppress primary or metastatic tumor growth; and will stop cancer progression, leading to stabilization of the disease.

(ii) Alternatively, if a greater than minimally effective concentration is administered daily to the human cancer patient, this even larger dosage and treatment regimen will cause a true regression of the cancer over time.

These in-vivo capabilities are demonstrated by the individual case histories of human cancer patients presented hereinafter.

5. It is also vital to understand and acknowledge that the present treatment methods are not limited by and are not contingent upon the water solubility characteristics of the chosen zinc salt in order to achieve therapeutic efficacy in-vivo. In particular, the present invention is neither based upon nor controlled by the ability of the chosen zinc containing medicament to dissolve in water and concomitantly release free zinc ions into an aqueous fluid. To the contrary, a wide range of slightly water soluble, hardly water soluble, and water insoluble zinc salts are preferentially and desirably utilized for therapeutic treatment purposes; and a broad variety of different biocompatible hydrocarbon and lipid based carrier compositions may be routinely employed in lieu of any aqueous-based carrier.

6. The present therapeutic methods do not demand and do not depend upon a single mechanism of action or biological activity. Unlike its predecessors in the prior art, the instant methodology intends and expects that a plurality of different and unrelated mechanisms of biological action will be operative concurrently in-vivo; and that such multiple mechanisms of biological assimilation and activity will be in effect, individually and collectively, within the living body as a consequence of administering a simple organic or inorganic salt of zinc orally, parenterally and/or via a body cavity routing to a cancer patient.

All of these alternative mechanisms of action will act cumulatively and thereby provide substantial in-vivo therapeutic efficacy for a variety of different zinc containing compounds that are either only slightly soluble, hardly soluble, or are substantially insoluble in aqueous based fluids. The overall result and sum effect of these multiple mechanisms of action is a general uptake and assimilation of various simple zinc salts by the dynamic systems intrinsically present in-vivo within the human body, which then serve to convey and deliver zinc to the cancer cells.

7. The existence of such multiple alternative mechanisms of action in-vivo for cancer patients is amply demonstrated and evidenced via several human case histories which employed zinc picolinate (which is only slightly soluble in water) as the chosen simple organic salt of zinc for therapeutic treatment purposes (see clinical case histories nos. 1-3 respectively presented hereinafter). The use of simple zinc salts which are either only slightly soluble or are substantially insoluble in water and aqueous based fluids as efficacious anti-cancer agents in-vivo is presently unknown and is contrary to the beliefs and expectations of the ordinary practitioner working in this technical field today.

### VII. Optional (Non-Obligatory) Concurrent Treatments

The simple organic and inorganic zinc salts described herein and exemplified by Table 2 above may be optionally combined and/or concurrently administered with one more chemotherapeutic drugs used today for the treatment of human cancers. However, in such optional and purely discretionary concurrent use circumstances, it is again emphasized and clearly understood that the simple zinc salt is always the principal anti-cancer agent; and that the administration of one or more simple zinc salt does not serve any other purpose or goal in-vivo. Specifically, the administration of the simple zinc salt does not enhance, nor activate, nor potentiate, nor aid any conventionally known chemotherapeutic drug which may then be present in admixture and/or administered concurrently.

Consequently, the administration of any conventionally known chemotherapeutic drug which might concurrently be given to the patient will not influence or functionally relate to the efficacy of the present treatment methodology so long as at least a minimally effective concentration of the chosen simple zinc salt is administered to the cancer patient.

### Optional Co-Administration with Chemotherapeutic Substances

The completely discretionary, non-compulsory, and freely elective co-administration of a conventional chemotherapeutic substance may be accomplished by intra-arterial, intravenous, intraperitoneal, intramuscular, and subcutaneous infusions and injections; by oral ingestion; and by rectal or vaginal insertion. For many cancer patients, an oral administration is preferred. However, for those cancer patients afflicted with obstruction of gastrointestinal tract by esophageal cancer, stomach cancer, colon cancer, pancreatic cancer, liver cancer, or other primary or metastatic tumors, an intravenous or body cavity route of administration is more desirable.

A representative listing of some conventionally known and used cancer chemotherapeutic substances today includes, but is not limited to, the substances listed by Table 3 below.

### Table 3: Conventional Cancer Chemotherapeutic Substances

Category 1. An alkylating agent such as nitrogen mustard, ethyleneimine, methylmelamine, mechlorethamine, melphalan, chlorambucil, busulfan, cyclophosphomide, ifosfamide, dacarbazine, procarbazine, nitrosoureas (carmustine, lomustine), alkyl sulfonate, triazene, cisplatin, carboplatin, oxaliplatin, thiotepa, and temozolamide.

Category 2. An antimetabolite such as methotrexate.

Category 3. A nucleotide analog such as mercaptopurine, thioguanine, fludarabine, hydroxyurea, cladribine, 5-fluorouracil, capecitabine, gemcitabine, cytarabine, azacitidine, azathioprine, and doxifluritidine.

Category 4. A plant derivative such as vinca alkaloids (vincristine, vinblastine, vindesin, vinorelbine), podophyllotoxins (etoposide, teniposide, tafluposide), and taxanes (paclitaxel, docetaxel).

Category 5. An antibiotic such as daunorubicin, doxorubicin, epirubicin, idarubicin, mitoxantrone, valrubicin, plicamycin, mitomycin, bleomycin, irinotecan, and dactinomycin.

Category 6. An endocrine agent such as tamoxifen, glucocorticoids, and flutamide.

Category 7. A biological response modifier such as all-trans-retinoic acid (ATRA), antibodies (cetuximab, bevacizumab, ibritumomab tiuxetan, rituximab, trastuzumab, gemtuzumab, alemtuzumab, centuximab, tositumomab, mitumomab, epratuzumab, edrecolomab, pentumomab), interleukin-2, interferon-alpha, and imatinib mesylate.

Category 8. Small molecule inhibitors such as imatinib mesylate (Glivec), geftinib (Iressa), Erlotinib (Tarceva), sunitinib (Sutent), sorafenib (Nexavar), bortezomib (Velcade), and axitinib.

Category 9. A miscellaneous agent such as anthracenedione and methyl hydrazine derivatives.

For a more detailed discussion and review of the various conventionally known cancer chemotherapeutic substances, their target diseases, and treatment protocols, see Goodman and Gilman's The Pharmacological Basis of Therapeutics, Ninth Ed., Pergamon Press, Inc., 1990; and Remington: The Science and Practice of Pharmacy, Mack Publishing Co., Easton, Pa., 1995.

### Optional Co-Administration with Ionizing Radiation

Another purely discretionary and non-compulsory cancer treatment for humans is the use of ionizing radiation. Under such circumstances, the present therapeutic treatment method—which administers not less than a minimally effective concentration of simple organic and inorganic salts of zinc—may be performed either before, or at the same time as, or after treatment with ionizing radiation.

### VIII. Human Patient Case Histories

To demonstrate the merits and value of the present invention, a variety of human case histories and clinical data are presented below. It will be expressly understood, however, that the clinical facts and data provided below are merely the best evidence of the subject matter as a whole which is the present invention; and that these human case studies are only illustrative of the full scope of the present invention as envisioned and claimed.

### Clinical Case History 1

A 73-year-old man was admitted to hospital with a cough, life threatening hemoptysis on Jan. 30, 2009. Bronchoscopy revealed a large and actively bleeding hemorrhagic mass obstructing the right bronchus intermedius; and the biopsy report showed a squamous cell carcinoma of the lung. A previous computer tomography examination of the patient (conducted on Jan. 14, 2009) revealed 3.1×2.3 cm soft tissue intrahilar mass within the right lower lobe, enlarged subcarinal lymph nodes, and right pleural effusion. Because the patient denied either operation or chemotherapy, his survival prognosis was poor, up to 2 months survival time.

The patient then began taking Zinc Picolinate, orally, at a dose concentration of 200 mg a day. The patient orally ingested Zinc Picolinate daily, for 5 months (from February, 2009 to July, 2009) at a concentration of 200 mg per day, as a dose of 100 mg ingested twice a day with a meal. To date, the patient has not taken any other chemotherapeutic anticancer drug/agent, nor has undergone any other form of conventional cancer treatment.

The patient has been orally ingesting 200 mg of Zinc Picolinate per day for nearly six calendar months time. The most recent computer tomography examination of patient's chest (Jul. 1, 2009) showed no right pleural infusion, no substantive changes in the size of primary lung tumor, and no meaningful changes in the size of enlarged subcarinal lymph nodes when compared to the previous computer tomography examination (performed on Jan. 14, 2009) when the initial diagnosis of lung tumor was made. The patient has been orally taking 600 mg of Zinc Picolinate daily from August, 2009 to October, 2009.

In this particular case of human lung cancer, the Zinc Picolinate treatment clinically led to a suppression of tumor growth, resulting in a stabilization of the disease for the patient. Equally important, at present (November, 2009) this patient has had no weight loss, no signs of cancer progression, and has not complained of any changes in his life style over the nine months of therapeutic treatment regimen with Zinc Picolinate. No toxic effects of Zinc Picolinate treatment were observed.

### Clinical Case History 2

A 28-year-old woman, has had a hyperpigmented area on her right thigh since childhood, which was presumed to be a benign congenital nevus. In November 2008, the skin lesion became progressively larger and ulcerated. Then in December 2008, a skin tumor resection and dissection of an enlarged right inguinal lymph nodes were performed, yielding melanoma and inguinal lymph nodes with metastatic disease.

In January 2009, a course of conventional chemotherapy was prescribed and performed for the patient. In February 2009, the patient had a cancer recurrence and progression, evidenced by tumor growth in the area of the resected right inguinal lymph node.

Because the metastatic lesion extended from her right inguinal region toward right knee along anterior surface of thigh was estimated as being 16×8 cm (February, 2009) and had a density described by surgeon as “stone hard” pressed femoral blood vessels, the patient had significant edema of her right leg. Enlarged nodes were then detected in the patient's abdominal cavity by ultrasound examination, suggesting peritoneal metastatic disease. The patient was subsequently discharged from the hospital, with a poor prognosis (up to 2 months survival time).

The patient then began orally taking Zinc Picolinate at a 600 mg per day dosage. She orally ingested Zinc Picolinate daily, for 5 months time (from February, 2009 to May, 2009); and then Zinc Sulfate daily for 1 month (June, 2009) at a dose of 600 mg per day (300 mg twice a day with a meal) and for 4 months (from July, 2009 to October, 2009) at a dose of 900 mg per 24 hours (300 mg every 8 hours with a meal).

In July 2009, a follow-up medical examination revealed a significant decrease in the size of metastatic lesion on patient's right leg, from 16×8 cm (as measured in February, 2009) to 8×4 cm. Examination also showed that the edema of the patient's right leg significantly decreased.

Using computer tomography and ultrasound examination, the most recent medical examination (Oct. 20, 2009) showed a complete regression of metastatic nodes in abdominal cavity and a significant regression of metastatic lesion on patient's right leg to 2×1 cm.

The patient was also specifically examined for clinical manifestations of zinc toxicity, but no toxic effects of zinc treatment were observed. To date, the patient has no weight loss and has not complained about any changes in her life style.

### Clinical Case History 3

A 57-year-old man afflicted with lung cancer (tumor stenosis of right upper lobe bronchus, squamous cell carcinoma) had been conventionally treated with radiotherapy. Nevertheless, his tumor still showed multiple granulations, bleeding erosions, and an irregular shape with bronchus obstruction (90% of bronchial lumen was obstructed)—as observed on bronchoscopy examination (performed on Jun. 14, 2009).

The patient then began to take Zinc Picolinate daily, for 1 month, at a dose of 600 mg per day (orally ingested as 300 mg of Zinc Picolinate taken twice a day with meals). After one month's treatment time with Zinc Picolinate, an X-ray examination showed a 2-fold decrease in the size of the lung tumor. The patient continued to take Zinc Picolinate orally from August, 2009 till October, 2009—but at a dose of 900 mg per day (orally ingested as 300 mg of Zinc Picolinate every 8 hours with meals).

The most recent bronchoscopy (performed on Oct. 21, 2009) revealed a significant decrease in tumor size, and a complete opening of the right upper lobe bronchus (previously obstructed by tumor) with benign changes in tumor appearance. Moreover, the patient noticed a significant improvement of his overall physical condition with loss of such symptoms as cough and shortness of breath. A physical examination revealed that there was no weight loss as such, and that no zinc toxic effects were clinically observed in the patient.

### Overview and Significance

A. It is of particular importance to note that Zinc Picolinate was taken by three cancer patients (Clinical Case Histories 1-3) for the treatment of cancer in high doses ranging from 200 mg to 900 mg per day, daily for 4-9 months and no zinc toxicity was observed. The patient with lung cancer (Clinical Case History 3) was taking Zinc Picolinate at a dose of 600 mg per day, daily for 1 month, and 900 mg per day, daily for 3 months, and this patient had a significant improvement of his general condition with no toxic effects of zinc. The patient with metastatic melanoma (Clinical Case History 2) was taking Zinc Picolinate at a dose of 600 mg per day, daily for 5 months, and 900 mg per day, daily for 4 months, and this patient had no toxic effects of Zinc Picolinate. Based on such clinical evidence, the present therapeutic methods are deemed to be safe and efficacious in-vivo for the treatment of different human cancers.

B. As shown by Clinical Case History 1, a lung cancer patient orally ingested Zinc Picolinate daily, for 5 months at a dose of 200 mg per day. The patient was not given any other chemotherapeutic anticancer drug or any other form of cancer treatment. Yet, in this particular case, the administration of 100 mg of Zinc Picolinate twice a day (i.e., the minimally effective concentration of 200 mg daily) led to suppression of tumor growth and stabilization of the disease. In addition, because no toxic effects resulted from zinc salt treatment was observed in this patient, such a minimally effective concentration of a zinc salt might be recommended for long term treatment of cancer patients as “tumor dormancy” therapy.

C. As shown by Clinical Case History 3, a lung cancer patient orally ingested 300 mg of Zinc Picolinate twice a day for 1 month (i.e., a preferred concentration of in the dose of 600 mg per day). After only one month of such Zinc Picolinate therapy, X-ray examination showed a 2-fold decrease in tumor size. The patient continued to take Zinc Picolinate from August, 2009 till October, 2009 at a dose of 900 mg per day (ingested as 300 mg of Zinc Picolinate every 8 hours with meal). The most recent bronchoscopy (performed on Oct. 21, 2009) revealed a significant decrease in tumor size, complete opening of the right upper lobe bronchus (previously obstructed by tumor) with benign changes in tumor appearance. The patient noticed a significant improvement of his overall physical condition with loss of such symptoms as cough and shortness of breath. Moreover, despite daily intake of this large dosage of zinc salt, no toxic effects of zinc treatment were observed in this patient.

Also, as shown by Clinical Case History 2, a cancer patient with melanoma metastasis ingested Zinc Picolinate daily, for 5 months time (from February, 2009 to June, 2009) in the dose of 600 mg per day (as 300 mg twice a day with meal) and for 4 months (from July, 2009 to October, 2009) in the dose of 900 mg per 24 hours (as 300 mg every 8 hours with meal). Using computer tomography and ultrasound examination, most recent medical examination (Oct. 20, 2009) showed a complete regression of metastatic nodes in abdominal cavity and a significant regression of metastatic lesion on patient's right leg from 16×8 cm (February, 2009) to 2×1 cm (October, 2009).

Thus, based on such clinical results (Case History 2 and 3), a preferred effective concentration of 600 to 1,000 mg of a zinc salt is recommended in order to induce primary or metastatic tumor regression in-vivo.

The present invention is not restricted in form nor limited in scope except by the claims appended hereto:

What I claim is:

1. An oral method for therapeutically treating a living human subject afflicted with a recognized type of cancer, said oral therapeutic treatment method comprising the steps of:
obtaining an orally ingestible preparation comprising a zinc compound selected from the group consisting of simple inorganic salts of zinc and simple organic salts of zinc and a biocompatible carrier composition suitable for oral ingestion;
orally administering not less than about 200 milligrams of said zinc compound preparation as an anti-cancer medicament to the afflicted human subject over 24 hours time;
repeating said oral administration of said zinc compound preparation as an anti-cancer medicament to the afflicted human subject every day as a therapeutic regimen for at least an initially scheduled treatment time;
periodically monitoring the status of the afflicted human subject during said oral treatment regimen to confirm that manifestations of zinc toxicity have not appeared; and
clinically determining that said oral treatment regimen using said zinc compound preparation as an anti-cancer medicament has been effective and that the recognized type of cancer at least has become demonstrably stabilized in the afflicted human subject.

2. A parenteral method for therapeutically treating a living human subject afflicted with a recognized type of cancer, said parenteral therapeutic treatment method comprising the steps of:
obtaining a sterile preparation comprising a zinc compound selected from the group consisting of simple inorganic salts of zinc and simple organic salts of zinc and an aqueous carrier fluid suitable for parenteral administration;
parenterally administering not less than about 0.5 milligrams per kilogram of body weight of said zinc compound preparation as an anti-cancer medicament to the afflicted human subject over 24 hours time;
repeating said parenteral administration of said zinc compound preparation as an anti-cancer medicament to the afflicted human subject every day as a therapeutic regimen for at least an initially scheduled number of treatment months;
periodically monitoring the status of the afflicted human subject during said parenteral treatment regimen to confirm that manifestations of zinc toxicity have not appeared; and
clinically determining that said parenteral treatment regimen using said zinc compound preparation as an anti-cancer medicament has been effective and that the recognized type of cancer at least has become demonstrably stabilized in the afflicted human subject.

3. The method for therapeutically treating a living human subject afflicted with a recognized type of cancer as recited in claim 2 wherein the form of said parenteral administration is one selected from the group consisting of intra-arterial, intravenous, intraperitoneal, intramuscular, intradermal, intratumoral and subcutaneous routes.

4. A body cavity method of therapeutically treating a living human subject afflicted with a recognized type of cancer, said body cavity therapeutic treatment method comprising the steps of:
obtaining a body cavity acceptable preparation comprising a zinc compound selected from the group consisting of simple inorganic salts of zinc and simple organic salts of zinc and a biocompatible carrier suitable for administration to a body cavity in an afflicted human subject;
administering not less than about 0.5 milligrams per kilogram of body weight of said zinc compound preparation as an anti-cancer medicament to a body cavity of the afflicted human subject over 24 hours time;
repeating said body cavity administration of said zinc compound preparation as an anti-cancer medicament to the afflicted human subject every day as a therapeutic regimen for at least an initially scheduled treatment time;
periodically monitoring the status of the afflicted human subject during said body cavity treatment regimen to confirm that manifestations of zinc toxicity have not appeared; and
clinically determining that said body cavity treatment regimen using said zinc compound preparation has been effective and that the recognized type of cancer at least has become demonstrably stabilized in the afflicted human subject.

5. The method for therapeutically treating a living human subject afflicted with a recognized type of cancer as recited in claim 4 wherein the form of said body cavity administration is one selected from the group consisting of abdominal, thoracic, rectal and vaginal administrations.

6. The method for therapeutically treating a living human subject afflicted with a recognized type of cancer as recited in claim 1, 2, or 4 wherein said administration of said zinc compound results in a clinical determination that the recognized type of cancer has regressed.

7. The method for therapeutically treating a living human subject afflicted with a recognized type of cancer as recited in claim 1, 2, or 4 wherein said administration of said zinc compound results in a clinical determination that the recognized type of cancer has been eliminated.

8. The method for therapeutically treating a living human subject afflicted with a recognized type of cancer as recited in claim 1, 2, or 4 wherein said administration of said zinc compound is performed from two to five times over each 24 hours time.

9. The method for therapeutically treating a living human subject afflicted with a recognized type of cancer as recited in claim 1, 2, or 4 wherein said administration of said zinc compound is made in the absence of any other anti-cancer medicament,

10. The method for therapeutically treating a living human subject afflicted with a recognized type of cancer as recited in claim 1, 2, or 4 wherein said zinc compound is zinc picolinate.

11. The method for therapeutically treating a living human subject afflicted with a recognized type of cancer as recited in claim 1, 2, or 4 wherein said zinc compound is one selected from the group consisting of zinc sulfate, zinc gluconate, zinc acetate, zinc glycinate zinc methionine sulfate, zinc chloride, zinc phosphate, zinc carbonate, zinc monomethionine, zinc pyrithione, zinc acexamate, zinc ascorbate, zinc aspartate, zinc citrate, zinc orotate, zinc glycerate, zinc salicylate, zinc propionate, and zinc lactate.

12. The method for therapeutically treating a living human subject afflicted with a recognized type of cancer as recited in claim 1, 2 or 4 wherein said zinc compound preparation is a solid.

13. The method for therapeutically treating a living human subject afflicted with a recognized type of cancer as recited in claim 1, 2, or 4 wherein said zinc compound preparation is a liquid.
